(19) United States
(12) Patent Application Publication
(10) Pub. No.: US 2008/0319019 A1
(43) Pub. Date: Dec. 25, 2008

(54) Therapeutic Agents

(76) Inventor: Leifeng Cheng, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 11/793,375

(22) PCT Filed: Dec. 21, 2005
(86) PCT No.: PCT/GB05/04956

§ 371 Date: Aug. 25, 2008

(30) Foreign Application Priority Data
Dec. 23, 2004 (GB) 0428073.1
Jul. 13, 2005 (GB) 0514348.2

(51) Int. Cl. A61K 31/4164; A61P 3/04; A61P 25/00; C07D 401/02; A61K 31/454; C07D 233/90

(52) U.S. Cl. 514/326; 548/333.5; 514/400; 546/210

(57) ABSTRACT

The present invention relates to certain 1,2-diarylimidazoles of formula I

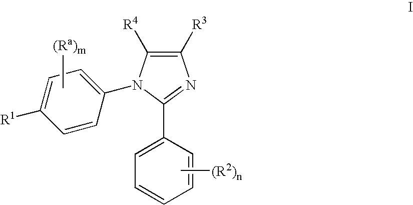

and processes for preparing such compounds, their use in the treatment of obesity, psychiatric and neurological disorders, to methods for their therapeutic use and to pharmaceutical compositions containing them.

### FIELD OF INVENTION

The present invention relates to certain 1,2-diarylimidazoles of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WO01/70700 and EP 656354).

WO04/60367 discloses that certain diaryl imidazoles and triazoles are useful as COX-1 inhibitors useful in the treatment of inflammation. DD 140966 discloses that certain imidazolecarboxylic acid anilides are useful as plant growth regulators.

WO 03/007887 and WO03/075660 disclose certain 4,5-diarylimidazole-2-carboxamides as CB₁ modulators.

J. Med. Chem. 2005, 48, 1823, WO03/27076, WO 2004/052864 and WO 03/63781 disclose certain 1,2-diarylimidazole-4-carboxamides which are CB₁ modulators.

WO03/40107 discloses certain 1,2-diarylimidazole-4-carboxamides as being useful in the treatment of obesity and obesity-related disorders.

Co-pending application WO2005/095354 discloses 4-[2-(substituted phenyl)-3-[(carboxamido]imidazol-1-yl]phenyl 1-alkanesulfonic acid ester derivatives as having CB₁ modulatory activity.

However, there is a need for CB₁ modulators with improved physicochemical properties and/or DMPK properties and/or pharmacodynamic properties.

### DESCRIPTION OF THE INVENTION

The invention relates to a compound of formula (I)

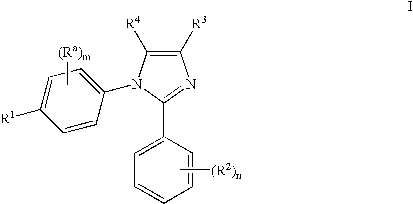

and pharmaceutically acceptable salts thereof, in which
R¹ represents a) a C₁₋₆alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;
R^{a} represents halo, a C₁₋₃alkyl group or a C₁₋₃alkoxy group;
n is 0, 1, 2 or 3;
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸ in which X is CO or SO₂,
Y is absent or represents NH optionally substituted by a C₁₋₃alkyl group; and R⁷ and R⁸ independently represent:
a C₁₋₉alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl), in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜ Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents carboxy, a C₁₋₃alkoxycarbonyl group, hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl.

In a particular group of compounds of formula (I), R³ represents

a) a group X—Y—NR⁷R⁸
in which X is CO or SO₂,
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl), in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜ Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;

wherein the heterocyclic group is optionally substituted by one or more C₁₋₃alkyl groups, hydroxy, fluoro or benzyl;

or b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl,
each optionally substituted by 1, 2 or 3 groups Z.

In a particular group of compounds of formula (I), W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl.

In a particular group of compounds of formula (I)

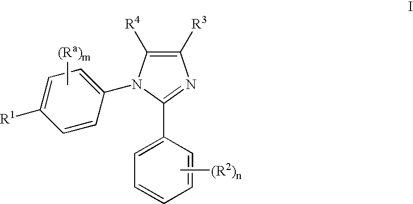

and pharmaceutically acceptable salts thereof,
R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O in which R⁵ represents a C₁₋₆alkyl group optionally substituted by one or more fluoro, or R⁵ represents phenyl or a heteroaryl group each of which is optionally substituted by 1, 2 or 3 groups represented by Z;
R^{a} represents halo, a C₁₋₃alkyl group or a C₁₋₃alkoxy group;
m is 0, 1, 2 or 3;
R² represents halo
n is 0, 1, 2 or 3;
R³ represents
a) a group X—Y—NR⁷R⁸ in which X is CO;
Y is absent or represents NH optionally substituted by a C₁₋₃alkyl group;
and R⁷ and R⁸ independently represent:
a C₁₋₆alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂), Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen;
wherein the heterocyclic group is optionally substituted by one or more C₁₋₃alkyl groups, hydroxy, fluoro or benzyl;
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl.

In a particular group of compounds of formula I, R¹ represents a group R⁵S(O)₂O in which R⁵ represents a C₁₋₆alkyl group optionally substituted by one or more fluoro and in which R², R³, R⁴, R^{a}, m and n are as previously defined.

In a particular group of compounds of formula I, R³ represents a group CONHNR⁷R⁸ in which NR⁷R⁸ represents piperidino.

It will be understood that where a substituent Z is present in more than one group that these substituents are independently selected and may be the same or different. The same is true for W. Similarly when m is 2 or 3 then the groups R^{a} are independently selected so that they may be the same or different and similarly when n is 2 or 3 then the groups R² are independently selected so that they may be the same or different. Similarly when R⁵ and R⁷ and/or R⁸ contain a heteroaryl group the heteroaryl groups and their optional substituents are independently selected so that they may be the same or different.

The term C₃₋₁₅cycloalkyl includes monocyclic, bicyclic, tricyclic and spiro systems for example, cyclopentyl, cyclohexyl and adamantyl.

The term heteroaryl means an aromatic 5-, 6-, or 7-membered monocyclic ring or a 9- or 10-membered bicyclic ring, with up to five ring heteroatoms selected from oxygen, nitrogen and sulfur. Suitable aromatic heteroaryl groups include, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl. Preferably furyl, pyrrolyl, thienyl, oxazolyl, isoxazolyl, imidazolyl, pyrazolyl, oxazolyl thiazolyl, isothiazolyl, oxadiazolyl, thiadiazolyl, triazolyl, tetrazolyl, pyridyl, pyridazinyl, pyrimidinyl, pyrazinyl or 1,3,5-triazenyl and more preferably pyrrolyl, thienyl, imidazolyl, oxazolyl or pyridyl. Suitable saturated or partially unsaturated 5 to 8 membered heterocyclic groups containing one or more heteroatoms selected from nitrogen, oxygen or sulphur include, for example tetrahydrofuranyl, tetrahydropyranyl, 2,3-dihydro-1,3-thiazolyl, 1,3-thiazolidinyl, pyrrolinyl, pyrrolidinyl, morpholinyl, tetrahydro-1,4-thiazinyl, 1-oxotetrahydrothienyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl or tetrahydropyrimidinyl, preferably tetrahydrofuranyl, tetrahydropyranyl, pyrrolidinyl, morpholinyl, piperidinyl or piperazinyl, more preferably tetrahydrofuran-3-yl, tetrahydropyran-4-yl, pyrrolidin-3-yl, morpholino, piperidino, piperidin-4-yl or piperazin-1-yl.

A particular group of compounds of formula I is represented by formula IA

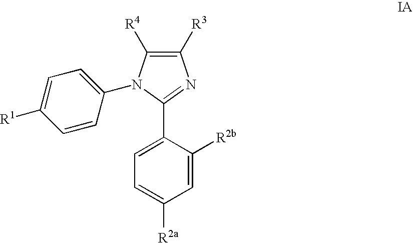

in which R¹ is
a) a C₃₋₆alkoxy group substituted by one or more fluoro, b) a group of formula phenyl(CH₂)ₚO— in which p is 1, 2 or 3 and the phenyl ring is optionally substituted by 1, 2 or 3 groups represented by Z, c) a group R⁵S(O)₂O in which R⁵ represents a C₁₋₆alkyl group optionally substituted by one or more fluoro;
R^{2a} represents H or chloro;
R^{2b} represents H or chloro;
R³ represents a group CONHNR⁷R⁸ in which NR⁷R⁸ represents piperidino; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;

Another particular group of compounds of formula I is represented by formula IA

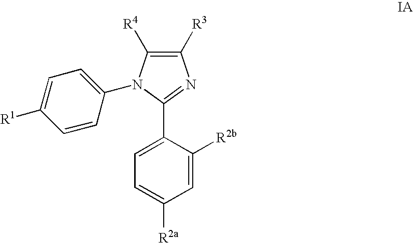

in which R¹ is
a) a C₃₋₆alkoxy group substituted by one or more fluoro or b) a group R⁵S(O)₂O in which R⁵ represents a C₁₋₆alkyl group optionally substituted by one or more fluoro, or R⁵ represents phenyl or a heteroaryl group each of which is optionally substituted by 1, 2 or 3 groups represented by Z;
R^{2a} represents chloro;
R^{2b} represents chloro;
R³ represents a group CONHNR⁷R⁸ in which NR⁷R⁸ represents piperidino; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

Another particular group of compounds of formula I is represented by formula IB

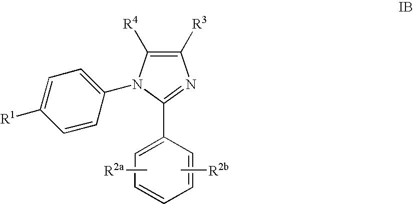

in which R¹ is
a) a C₃₋₆alkoxy group substituted by one or more fluoro or b) a group R⁵S(O)₂O in which
R⁵ represents a C₁₋₆alkyl group optionally substituted by one or more fluoro;
R^{2a} represents H, a C₁₋₃alkyl group, chloro, fluoro or cyano;
R^{2b} represents a C₁₋₃alkyl group, chloro, fluoro or cyano;
R³ represents a group CONHNR⁷R⁸ in which NR⁷R⁸ represents piperidino or R³ represents a group CONHR⁸ in which R⁸ represents a cycloalkyl group optionally substituted by one or more of the following: fluoro, hydroxy, a group NR^{e}R^{f} in which R^{e} and R^{f} independently represent H or a C₁₋₆alkyl group or R⁸ represents a C₅₋₈alkyl group optionally substituted by hydroxy; and
R⁴ represents a C₁₋₃alkyl group substituted at the terminal carbon by hydroxy.

By terminal it will be understood that the hydroxy group is attached to the ω (omega) position that is the carbon in the alkyl chain furthest from the point of attachment to the imidazole ring eg hydroxymethyl, 2-hydroxyethyl or 3-hydroxypropyl.

In a particular group of compounds of formula IA, R¹ represents a group R⁵S(O)₂O in which R⁵ represents a C₁₋₆alkyl group optionally substituted by one or more fluoro;

R^{2a} represents chloro;
R^{2b} represents chloro;
R³ represents a group CONHNR⁷R⁸ in which NR⁷R⁸ represents piperidino; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

Further values of R¹, R³ and R⁴ in compounds of formula I, formula IA and IB now follow. It will be understood that such values may be used where appropriate with any of the definitions, claims or embodiments defined hereinbefore or hereinafter.

In a particular group of compounds of formula I or formula IA R⁴ represents a group of formula CH₂NR^{e}R^{f} in which R^{e} and R^{f} are as previously defined.

In a further particular group of compounds of formula I, formula IA or formula IB, R⁴ represents a group of formula CH₂OH.

In a further particular group of compounds of formula I, formula IA or formula IB, R³ represents N-(piperidin-1-yl)carbamoyl.

In one group of compounds of formula I, formula IA or formula IB, R¹ is a C₃₋₆alkoxy group substituted by one or more fluoro. In a second group of compounds of formula I or formula IA, R¹ is a group of formula phenyl(CH₂)ₚO— in which p is 1, 2 or 3. In a third group of compounds of formula I, formula IA, or formula IB, R¹ is a group R⁵S(O)₂O in which R⁵ represents a C₁₋₆alkyl group optionally substituted by one or more fluoro. In a fourth group of compounds of formula I, formula IA, or formula IB, R¹ is a group R⁵S(O)₂O in which R⁵ represents a C₁₋₆alkyl group substituted by one or more fluoro. In a fifth group of compounds of formula I, formula IA, or formula IB, R¹ is a group R⁵S(O)₂O in which R⁵ represents a C₃₋₆alkyl group substituted by one or more fluoro. Particularly R¹ is 4,4,4-trifluorobutoxy, n-butylsulfonyloxy, n-propylsulfonyloxy, n-ethylsulfonyloxy, benzyloxy, 4,4,4-trifluorobutyl-1-sulfonyloxy, 3,3,3-trifluoropropyl-1-sulfonyloxy, 3-fluoropropoxy, 3,3,3-trifluoropropoxy or 3-fluoropropyl-1-sulfonyloxy. More particularly R¹ is 4,4,4-trifluorobutoxy, n-butylsulfonyloxy, n-propylsulfonyloxy, n-ethylsulfonyloxy, benzyloxy, 4,4,4-trifluorobutyl-1-sulfonyloxy or 3,3,3-trifluoropropyl-1-sulfonyloxy. Most particularly R¹ 3,3,3-trifluoropropyl-1-sulfonyloxy. 4,4,4-trifluorobutyl-1-sulfonyloxy or n-propylsulfonyloxy.

Particularly R³ is N-(piperidin-1-yl)carbamoyl, N-(4,4-difluorocyclohexyl)carbamoyl, N-cyclohexylcarbamoyl, N-(2-hydroxycyclohexyl)carbamoyl, N-(3-hydroxycyclohexyl)carbamoyl, N-(1-hydroxymethyl-3-methylbutyl)carbamoyl), N (1-ethylbutyl)carbamoyl, N-(2-aminocyclohexyl)carbamoyl, N-(1,4-dimethyl-pentyl)carbamoyl) or N-(3-dimethylaminocyclohexyl)carbamoyl. More particularly R³ is N-(piperidin-1-yl)carbamoyl, N-(4,4-difluorocyclohexyl)carbamoyl, N-cyclohexylcarbamoyl, or N-(2-hydroxycyclohexyl)carbamoyl. Most particularly R³ is N-(piperidin-1-yl)carbamoyl.

“Pharmaceutically acceptable salt”, where such salts are possible, includes both pharmaceutically acceptable acid and base addition salts. A suitable pharmaceutically acceptable salt of a compound of Formula I is, for example, an acid-addition salt of a compound of Formula I which is sufficiently basic, for example an acid-addition salt with an inorganic or organic acid or a base-addition salt of a compound of Formula I which is sufficiently acidic for example a base-addition salt with an inorganic base or an organic base.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl and t-butyl. Preferred alkyl groups are methyl, ethyl, propyl, isopropyl and tertiary butyl.

Unless otherwise stated or indicated, the term “alkoxy” denotes a group O-alkyl, wherein alkyl is as defined above.

Unless otherwise stated or indicated, the term “halogen” shall mean fluorine, chlorine, bromine or iodine.

Specific compounds of the invention are one or more of the following:
propane-1-sulfonic acid 4-[2-(2,4-dichlorophenyl)-5-hydroxymethyl-4-(piperidin-1-ylcarbamoyl)-imidazol-1-yl]phenyl ester;
3,3,3-trifluoropropane-1-sulfonic acid 4-[2-(2,4-dichlorophenyl)-5-hydroxymethyl-4-(piperidin-1-ylcarbamoyl)imidazol-1-yl]phenyl ester;
3,3,3-trifluoropropane-1-sulfonic acid 4-[2-(2,4-dichlorophenyl)-4-(4,4-difluoro-cyclohexylcarbamoyl)-5-hydroxymethylimidazol-1-yl]phenyl ester;
3,3,3-trifluoropropane-1-sulfonic acid 4-[4-cyclohexylcarbamoyl-2-(2,4-dichlorophenyl)-5-hydroxymethylimidazol-1-yl]-phenyl ester;
3,3,3-trifluoropropane-1-sulfonic acid 4-[2-(2,4-dichlorophenyl)-4-(cis-2-hydroxy-cyclohexylcarbamoyl)-5-hydroxymethyl-imidazol-1-yl]-phenyl ester;
3,3,3-trifluoropropane-1-sulfonic acid 4-[2-(3-cyano-5-fluoro-phenyl)-4-(4,4-difluoro-cyclohexylcarbamoyl)-5-hydroxymethylimidazol-1-yl]phenyl ester;
3,3,3-trifluoropropane-1-sulfonic acid 4-[2-(3-cyanophenyl)-5-hydroxymethyl-4-(1-hydroxymethyl-3-methylbutylcarbamoyl)imidazol-1-yl]phenyl ester;
3,3,3-trifluoro-propane-1-sulfonic acid 4-[4-(2-aminocyclohexylcarbamoyl)-2-(3-cyano-5-fluoro-phenyl)-5-hydroxymethylimidazol-1-yl]phenyl ester;
3,3,3-trifluoropropane-1-sulfonic acid 4-[2-(3-cyano-5-fluoro-phenyl)-4-(3-dimethylamino-cyclohexylcarbamoyl)-5-hydroxymethylimidazol-1-yl]phenyl ester;
3,3,3-trifluoropropane-1-sulfonic acid 4-[2-(3-cyano-5-fluoro-phenyl)-4-(2-hydroxy-cyclohexylcarbamoyl)-5-hydroxymethylimidazol-1-yl]phenyl ester;
3,3,3-trifluoropropane-1-sulfonic acid 4-[2-(3-cyanophenyl)-4-(2-hydroxy-cyclohexylcarbamoyl)-5-hydroxymethylimidazol-1-yl]phenyl ester;
3,3,3-trifluoropropane-1-sulfinic acid 4-[2-(3-cyano-5-fluorophenyl)-4-(3-hydroxy-cyclohexylcarbamoyl)-5-hydroxymethyl-imidazol-1-yl]phenyl ester;
2-(2,4-dichlorophenyl)-1-[4-(3-fluoropropoxy)phenyl]-5-hydroxymethyl-1H-imidazole-4-carboxylic acid cyclohexylamide;
3,3,3-trifluoropropane-1-sulfonic acid 4-[2-(2-chlorophenyl)-4-(2-hydroxy-cyclohexylcarbamoyl)-5-hydroxymethylimidazol-1-yl]phenyl ester;
3,3,3-trifluoropropane-1-sulfonic acid 4-[2-(2-chlorophenyl)-4-(1,4-dimethyl-pentylcarbamoyl)-5-hydroxymethyl-imidazol-1-yl]phenyl ester;
2-(2,4-dichlorophenyl)-5-hydroxymethyl-1-[4-(3,3,3-trifluoropropoxy)phenyl]-1H-imidazole-4-carboxylic acid (2-hydroxy-cyclohexyl)amide;
3,3,3-trifluoropropane-1-sulfonic acid 4-[2-(4-chloro-2-methyl-phenyl)-5-hydroxymethyl-4-(piperidin-1-ylcarbamoyl)-imidazol-1-yl]phenyl ester;
2-(2,4-dichlorophenyl)-5-hydroxymethyl-1-[4-(3,3,3-trifluoropropoxy)phenyl]-1H-imidazole-4-carboxylic acid (3-hydroxycyclohexyl)amide;
3-fluoro-propane-1-sulfonic acid 4-[2-(2,4-dichlorophenyl)-4-(2-hydroxy-cyclohexylcarbamoyl)-5-hydroxymethylimidazol-1-yl]phenyl ester;
4,4,4-trifluorobutane-1-sulfonic acid 4-[2-(3-cyano-5-fluorophenyl)-4-(1-ethyl-butylcarbamoyl)-5-hydroxymethyl-imidazol-1-yl]phenyl ester; and
3,3,3-trifluoropropane-1-sulfonic acid 4-[2-(2-chlorophenyl)-4-cyclohexylcarbamoyl-5-hydroxymethylimidazol-1-yl]phenyl ester;
   as well as pharmaceutically acceptable salts thereof. It will be understood that the term one or more means that any combination of any number between 1 and 21 of the above is compounds is included in the scope of the present application.

### Methods of Preparation

The compounds of the invention may be prepared as outlined below according to any of the following methods. However, the invention is not limited to these methods, the compounds may also be prepared as described for structurally related compounds in the prior art.

Compounds of formula I in which R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O may be prepared by reacting a compound of formula II

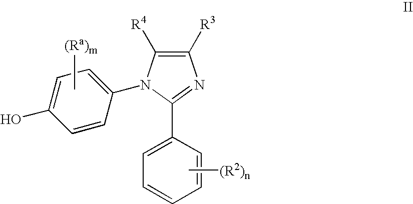

in which R², R³, R⁴, R^{a}, m and n are as previously defined with a group R^{1A}-X in which R^{1A} represents a group such that R^{1A}O represents R¹ and X represents a leaving group for example halo, at a temperature in the range of −25 to 150° C., in the presence of an inert solvent, for example dichloromethane, and optionally in the presence of a base for example triethylamine or pyridine.

Compounds of formula I in which R^{a}, R¹, R², R⁴, m and n are as previously defined and R³ represents a group X—Y—NR⁷R⁹ in which X is CO and Y, R⁷ and R⁸ are as previously defined may be prepared by reacting a compound of formula III

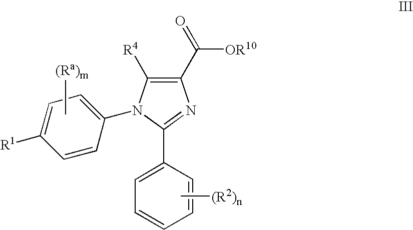

in which R^{a}, R¹, R², R⁴, m and n are as previously defined and R¹⁰ represents H or a C₁₋₆alkyl group with a compound of formula IV or a salt thereof

R⁷R⁸YNH₂ IV

in which Y, R⁷ and R⁸ are as previously defined, in an inert solvent, for example toluene, in the presence of a Lewis Acid, for example trimethylaluminium, at a temperature in the range of −25° C. to 150° C. when R¹⁰ is a C₁₋₆alkyl group; or alternatively when R¹³ is H by reacting a compound of formula III with a chlorinating agent for example oxalyl chloride, and then reacting the acid chloride produced with an amine of formula IV in an inert solvent, for example dichloromethane, in the presence of a base, for example triethylamine or pyridine, at a temperature in the range of −25° C. to 150° C.

Compounds of formula I in which R^{a}, R¹, R², R⁴, m and n are as previously defined and R³ represents a group X—Y—NR⁷R⁸ in which X is SO₂ may be prepared by reacting a compound of formula V

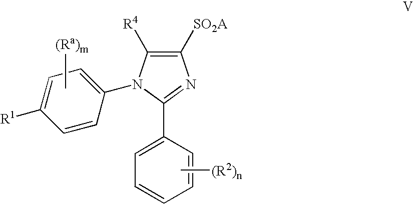

in which R^{a}, R¹, R², R⁴, m and n are as previously defined and A represents a leaving group, for example halo eg chloro, with a compound of formula IV in which Y, R⁷ and R⁸ are as previously defined or a salt thereof in an inert solvent, for example THF or dichloromethane, in the presence of a base, for example potassium carbonate, triethylamine or pyridine, at a temperature in the range of −25° C. to 150° C.

Compounds of formula I in which R^{a}, R², R³, R⁴, m and n are as previously defined and R¹ represents a group R⁵S(O)₂NH may be prepared by reacting a compound of formula VI

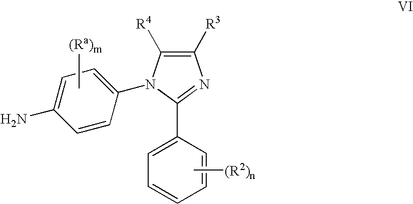

in which R^{a}, R², R³, R⁴, m and n are as previously defined with a sulphonating agent of formula R⁵SO₂L in which R⁵ is as previously defined and L represents a leaving group, for example chloro, in an inert solvent, for example dichloromethane, in the presence of a base, for example triethylamine, at a temperature in the range of −25° C. to 150° C.

Compounds of formula I in which R^{a}, R¹, R², R³, m and n are as previously defined and R⁴ represents OH may be prepared by reacting a compound of formula VII

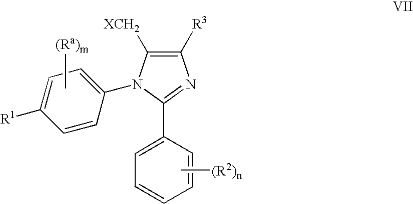

in which R^{a}, R¹, R², R³, m and n are as previously defined and X represents a leaving group for example halo e.g. bromo, chloro or iodo, with a hydrolysing agent for example silver nitrate in the presence of a solvent system for example aqueous acetone at a temperature in the range of 15-150° C.

Compounds of formula I in which R^{a}, R¹, R², R³, m and n are as previously defined and R⁴ represents CH₂NR^{e}R^{f} in which R^{e} and R^{f} are as previously defined may be prepared by reacting a compound of formula VII

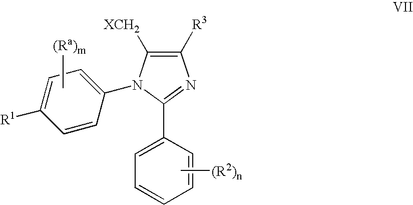

in which R^{a}, R¹, R², R³, m and n are as previously defined and X represents a leaving group for example halo e.g. bromo, chloro or iodo, with an amine of formula HNR^{e}R^{f} in which R^{e} and R^{f} are as previously defined in an inert solvent, for example ethanol, at a temperature in the range of 15-150° C.

Certain intermediate compounds of formula II, III, V, VI and VII are believed to be novel and form part of the present invention.

Compounds of formula II, III, V, VI and VII may be prepared by the general synthetic route shown at the end of the examples and adaptations thereof or by analogous methods known to those skilled in the art. It will be appreciated by those skilled in the art that during the reaction sequence certain functional groups, for example hydroxy groups and optionally substituted amino groups in R⁴, will require protection followed by deprotection at an appropriate stage see “Protective Groups in Organic Synthesis”, 3rd Edition (1999) by Greene and Wuts.

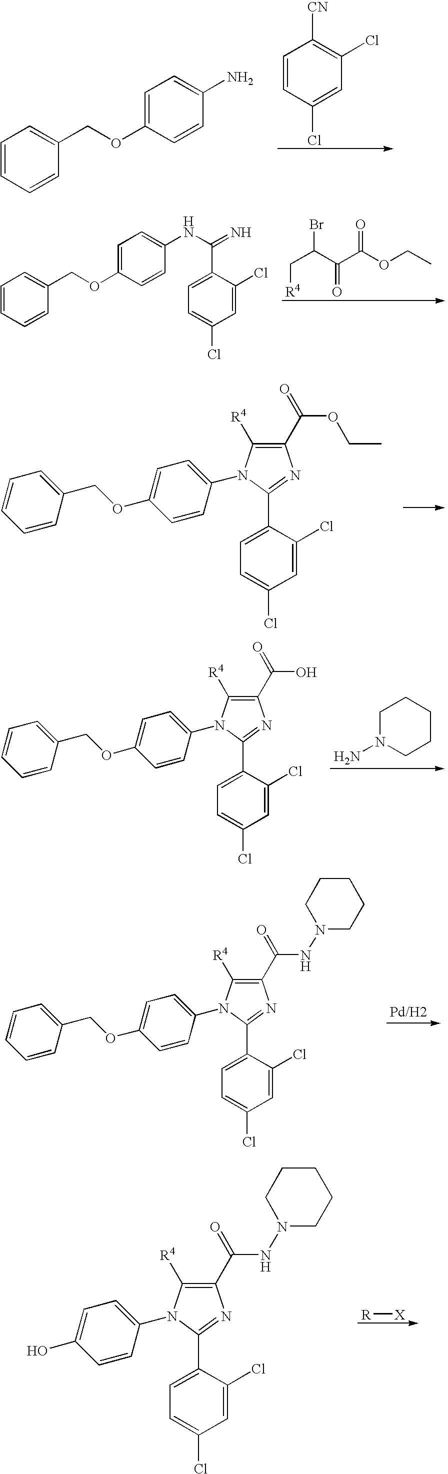

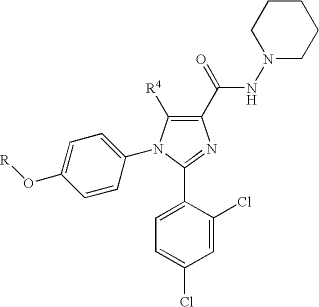

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disoerders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse. In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma. In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention are suitable for use in treating the above indications in juvenile or adolescent patient populations.

The compounds of the present invention may also be suitable for use in the regulation of bone mass and bone loss and therefore useful in the treatment of osteoporosis and other bone diseases.

The compounds of the present invention may also be used in the treatment of hepatic diseases, for example hepatic fibrosis, alcoholic liver cirrhosis, chronic viral hepatitis, non-alcoholic steatohepatitis or liver cancer.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier. According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man. According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment. Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

As the compounds of formula I are useful in causing smoking cessation, preventing weight gain resulting from smoking cessation, treating nicotine withdrawal and preventing nicotine dependence they may also be combined with other compounds known to have one or more of these effects for example nicotine, a nicotine agonist or a partial agonist, a monoamine oxidase inhibitor or antidepressants such as bupropion, doxepine, nortriptyline or an anxiolytic such as buspirone or clonidine.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)ÙD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar. For example the IC50 of Example 1 is 3 mM.

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example increasing the free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### EXAMPLES

### Abbreviations

AcOH acetic acid
AIBN 2,2′-azobisisobutyronitrile
BOP benzotriazol-1-yloxytris(dimethylamino)phosphonium hexafluorophosphate
DCM dichloromethane

### DEA Diethylamine

DMF dimethylformamide
DEA diethylamine

### DIEA N,N-diisopropylethylamine

DMAP 4-dimethylaminopyridine
EtOAc ethyl acetate
LiHMDS lithium hexamethyldisilazide
MeOH methanol
rt room temperature
THF tetrahydrofuran
TLC thin layer chromatography
t triplet
s singlet
d doublet
q quartet
qvint quintet
m multiplet
br broad
bs broad singlet
dm doublet of multiplet
bt broad triplet
dd doublet of doublet

### General Experimental Procedures

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC with a mass triggered fraction collector, Shimadzu QP 8000 single quadrupole mass spectrometer equipped with 19×100 mm C8 column. The mobile phase used was, if nothing else is stated, acetonitrile and buffer (0.1 M NH₄Ac:acetonitrile 95:5).

For isolation of isomers, a Kromasil CN E9344 (250×20 mm i.d.) column was used. Heptane:ethyl acetate:DEA 95:5:0.1 was used as mobile phase (1 ml/min). Fraction collection was guided using a UV-detector (330 nm).

### EXAMPLES OF THE INVENTION

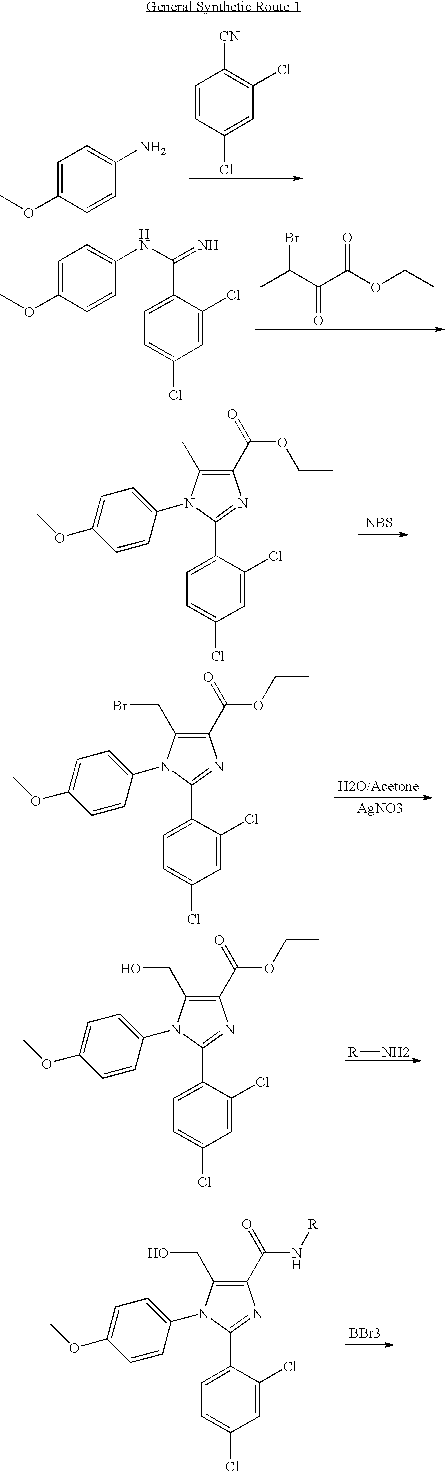

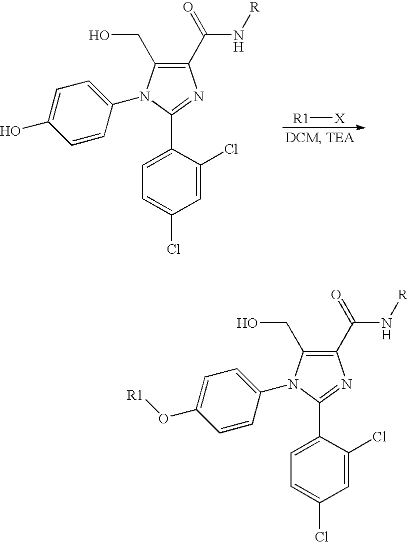

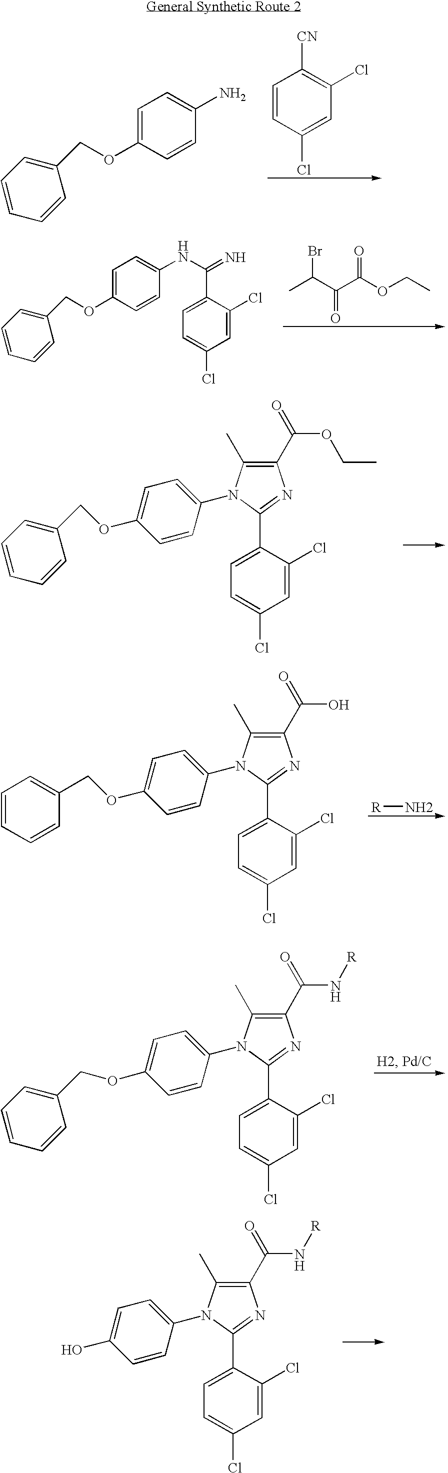

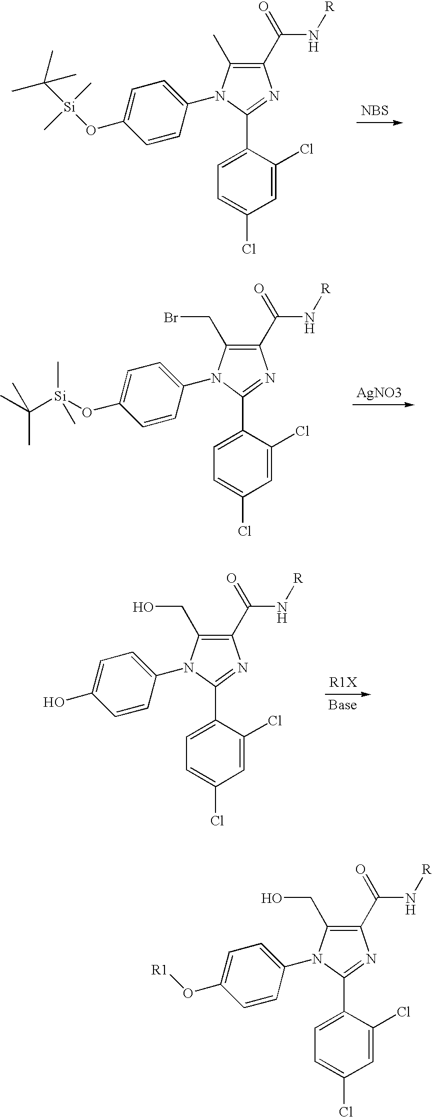

### Example 1

### Propane-1-sulfonic acid 4-[2-(2,4-dichlorophenyl)-5-hydroxymethyl-4-(piperidin-1-ylcarbamoyl)imidazol-1-yl]-phenyl ester

### Step A: (4-Methoxy-phenyl)-2,4-dichlorobenzamidine

p-Anisidine (6.16 g, 50 mmol) was added in portions to a solution of ethylmagnesium bromide (50 ml, 1M in THF, 50 mol) under a nitrogen atmosphere. After stirring for 30 minutes 2,4-dichlorobenzonitrile (8.60 g, 50 mmol) was added. The reaction mixture was stirred overnight at room temperature. Water (300 ml) was carefully added. The mixture was extracted with EtOAc (3×100 ml), dried (Na₂SO₄), filtered and evaporated to dryness to afford 14.1 g (100%) of the title compound used directly in the next step.

### Step B: 1-(4-Methoxyphenyl)-2-(2,4-dichlorophenyl)-5-methyl-1H-imidazole-4-carboxylic acid ethyl ester

To (4-methoxyphenyl)-2,4-dichloro-benzamidine (14.1 g, 0.05 mol) dissolved in 200 ml THF was added potassium carbonate (6.91 g, 0.05 mol) followed by slow addition of ethyl 3-bromo-2-oxo-butanoate (10.45 g, 0.05 mol) in 200 ml THF. The reaction mixture was stirred overnight at room temperature. The solution was filtered and evaporated to dryness. The residue was dissolved in acetic acid (150 ml) and boiled under reflux for 1 hour. The mixture was cooled to room temperature, water added and the product extracted with EtOAc (3×300 ml). The combined organic phases were washed with saturated sodium hydrogen carbonate, dried (Na₂SO₄), filtered and concentrated. Flash chromatography (heptane: EtOAc 70:30-50:50-EtOAc) afforded a crude product that was precipitated from hexane:EtOAc to afford 9.17 g (50%) of the title compound as a pale yellow solid.

### Step C: 5-Bromomethyl-2-(2,4-dichlorophenyl)-1-(4-methoxyphenyl)-1H-imidazole-4-carboxylic acid ethyl ester

To a magnetically stirred solution of 1-(4-methoxyphenyl)-2-(2,4-dichlorophenyl)-5-methyl-1H-imidazole-4-carboxylic acid ethyl ester (1.22 g, 3.00 mmol) in carbon tetrachloride (75 ml) was added N-bromosuccinimde (590 mg, 3.00 mmol) and 2,2′-azoisobutyronitrile (75 mg). The resulting mixture was refluxed for 1.5 hrs, cooled to room temperature and filtered. The solvent was removed under reduced pressure to give 1.46 g (100%) of the title compound as a pale yellow solid used directly in the next step.

### Step D: 2-(2,4-Dichlorophenyl)-5-hydroxymethyl-1-(4-methoxyphenyl)-1H-imidazole-4-carboxylic acid ethyl ester

To a suspension of 5-bromomethyl-2-(2,4-dichlorophenyl)-1-(4-methoxyphenyl)-1H-imidazole-4-carboxylic acid ethyl ester (1.46 g, 3.00 mmol) in 50% aqueous acetone (140 ml) was added silver nitrate (1.20 g, 7.00 mmol). The reaction mixture was stirred at 60° C. overnight, cooled to room temperature, water added and the product extracted with DCM (×3). The combined organic extracts were dried (Na₂SO₄), filtered and concentrated. Flash chromatography (Heptane:EtOAc 50:50) afforded 0.92 (73%) of the title compound as a colorless solid.

### Step E: 2-(2,4-Dichlorophenyl)-5-hydroxymethyl-1-(4-methoxyphenyl)-1H-imidazole-4-carboxylic acid piperidin-1-ylamide

To a magnetically stirred suspension of aluminium chloride (2.80 g, 20.99 mmol) in 1,2-dichloroethane (25 ml) was added 1-aminopiperidine (5 ml, 46 mmol) at 0° C. The suspension was allowed to warm to room temperature and a solution of 2-(2,4-dichloro-phenyl)-5-hydroxymethyl-1-(4-methoxyphenyl)-1H-imidazole-4-carboxylic acid ethyl ester (0.92 g, 2.18 mmol) in 1,2-dichloroethane (25 ml) was added. The reaction mixture was stirred overnight, water added and the product extracted with dichloromethane (×3). The combined organic extract was washed with water, dried (Na₂SO₄), filtered and concentrated. Flash chromatography (EtOAc-EtOAc:MeOH 80:20) afforded 1.00 g (96%) of the product as a colorless solid.

### Step F: 2-(2,4-Dichlorophenyl)-5-hydroxymethyl-1-(4-hydroxyphenyl)-1H-imidazole-4-carboxylic acid piperidin-1-ylamide

To a solution 2-(2,4-dichlorophenyl)-5-hydroxymethyl-1-(4-methoxyphenyl)-1H-imidazole-4-carboxylic acid piperidin-1-ylamide (0.98 g, 2.04 mmol) in dichloromethane (60 ml) at 0° C. was added boron tribromide (0.8 ml, 8.00 mmol). The cooling bath was removed and stirring continued for 3 hrs at 0° C. before pouring it onto ice-water and extracting with DCM (×3). The combined extracts were dried (Na₂SO₄), filtered and concentrated. Flash chromatography (Heptane:EtOAc gradient) afforded 0.57 g (61%) of the title compound as a colorless solid.

### Step G: Propane-1-sulfonic acid 4-[2-(2,4-dichlorophenyl)-5-hydroxymethyl-4-(piperidin-1-ylcarbamoyl)-imidazol-1-yl]phenyl ester

To a solution of 2-(2,4-dichlorophenyl)-5-hydroxymethyl-1-(4-hydroxyphenyl)-1H-imidazole-4-carboxylic acid piperidin-1-ylamide (0.43 g, 1.03 mmol) in dichloromethane (20 ml) was added triethylamine (0.14 ml, 1.04 mmol) and the reaction mixture cooled to 0° C. 1-Propanesulfonylchloride (0.10 ml, 1.03 mmol) was added, the cooling bath removed and the reaction mixture stirred at rt for 2 hrs. Water was added, the product extracted with DCM (×2), the combined organic extracts washed with water, dried (Na₂SO₄), filtered and concentrated. Flash chromatography (heptane:EtOAc gradient) afforded 200 mg (34%) of the title compound as a pale yellow solid. Recrystallisation (EtOAc) afforded 100 mg of a slightly yellow solid.

¹H NMR (CDCl₃): δ 7.50-7.10 (8H, m), 4.60 (2H, s), 3.30-3.20 (2H, m), 3.10-2.90 (4H, m), 2.10-1.80 (6H, s), 1.60-1.40 (2H, m), 1.08 (3H, t).

MS: 567 (M+H) 589 (M+Na). HPLC: 93%

### Example 2

### 3,3,3-Trifluoropropane-1-sulfonic acid 4-[2-(2,4-dichlorophenyl)-5-hydroxymethyl-4-(piperidin-1-ylcarbamoyl)imidazol-1-yl]phenyl ester

To a magnetically stirred solution of 2-(2,4-dichlorophenyl)-5-hydroxymethyl-1-(4-hydroxyphenyl)-1H-imidazole-4-carboxylic acid piperidin-1-ylamide, Ex. 1, Step F (410 mg, 0.89 mmol) in dry dichloromethane (10 ml) was added triethylamine (0.14 ml, 1.00 mmol) and the reaction mixture cooled to 0° C. 4,4,4-Trifluoropropanesulfonyl chloride (192 mg, 0.98 mmol) was added and the reaction mixture stirred for 4 hours at room temperature, poured into water and extracted with DCM (×3). The combined organic extract was dried (Na₂SO₄), filtered and concentrated. Flash chromatography afforded 280 mg of the title compound as a colorless solid; suspension of this material in heptane EtOAc gave 200 mg (36%) of the pure product.

¹H NMR (CDCl₃): δ7.50-7.10 (8H, m), 4.59 (2H, s), 3.60-3.45 (2H, m), 3.10-2.40 (7H, m), 1.90-1.70 (4H, m), 1.60-1.40 (2H, m).

### Example 3

### 3,3,3-Trifluoropropane-1-sulfonic acid 4-[2-(2,4-dichlorophenyl)-4-(4,4-difluoro-cyclohexylcarbamoyl)-5-hydroxymethylimidazol-1-yl]phenyl ester

### Step A

### N-(4-Benzyloxyphenyl)-2,4-dichlorobenzamidine

4-Benzyloxyaniline hydrochloride (20.0 g, 84.8 mmol) was added in portions to a solution of ethylmagnesium bromide (1 M in THF, 178 ml, 178 mmol) in dry THF (100 ml). After stirring for 30 min., 2,4-dichlorobenzonitrile (14.6 g, 84.9 mmol) was added. The reaction mixture was stirred at room temperature overnight, water was carefully added and the product extracted with EtOAc (×3). Drying (Na₂SO₄), filtration and concentration left 31.2 g (99%) of the title compound as a brown solid used directly in the next step.

### Step B

### 1-(4-Benzyloxyphenyl)-2-(2,4-dichlorophenyl)-5-methyl-1H-imidazole-4-carboxylic acid ethyl ester

To N-(4-benzyloxyphenyl)-2,4-dichlorobenzamidine (31.2 g, 84.0 mmol) dissolved in 110 ml THF was added potassium carbonate (11.7 g, 85.0 mmol) and the suspension was stirred for 10 minutes. Ethyl-3-bromo-2-oxobutanoate (21.4 g, 102 mmol) dissolved in THF (110 ml) was added slowly, and the mixture was stirred overnight at room temperature. The solution was filtered and evaporated to dryness. The residue was dissolved in acetic acid (200 ml) and boiled under reflux for 1 hour. The mixture was cooled to room temperature, water added and the product extracted with EtOAc (×3). The combined organic phases were washed with saturated sodium hydrogen carbonate, dried (Na₂SO₄), filtered and concentrated. Dry flash chromatography (silica, heptane:EtOAc gradient) followed by suspension in ether and filtration afforded 20.5 g (51%) of the title compound as a colourless solid.

### Step C

### 1-(4-Benzyloxyphenyl)-2-(2,4-dichlorophenyl)-5-methyl-1H-imidazole-4-carboxylic acid

To a suspension of 1-(4-benzyloxyphenyl)-2-(2,4-dichlorophenyl)-5-methyl-1H-imidazole-4-carboxylic acid ethyl ester (7.24 g, 15.0 mmol) in 120 ml methanol was added potassium hydroxide (8.10 g, 144 mmol) in water (40 ml), and the reaction mixture was boiled under reflux for 1 hour. The mixture was cooled to room temperature, acidified to pH-2 with 1 M HCl and extracted with ethyl acetate (×3). The combined organic phases were washed with brine, dried (Na₂SO₄), filtered and concentrated to give 6.80 g (100%) of the title compound.

### Step D

### 1-(4-Benzyloxyphonyl)-2-(2,4-dichlorophenyl)-5-methyl-1H-imidazole-4-carboxylic acid (4,4-difluorocyclohexyl)amide

To a solution of 1-(4-benzyloxyphenyl)-2-(2,4-dichlorophenyl)-5-methyl-1H-imidazole-4-carboxylic acid (1.80 g, 3.97 mmol) and 4,4-difluorocyclohexylamine (0.54 g, 3.99 mmol) was added triethylamine (1.40 ml, 10.0 mmol). BOP (2.12 g, 4.79 mmol) was added and the reaction mixture stirred overnight. Flash chromatography (heptane:EtOAc 70:30-50:50) gave 2.39 g (100%) of the title compound as a colorless solid.

### Step E

### 2-(2,4-Dichlorophenyl)-1-(4-hydroxyphenyl)-5-methyl-1H-imidazole-4-carboxylic acid (4,4-difluorocyclohexyl)amide

1-(4-Benzyloxyphenyl)-2-(2,4-dichlorophenyl)-5-methyl-1H-imidazole-4-carboxylic acid (4,4-difluorocyclohexyl)amide (2.39 g, 4.18 mmol) in ethanol (40 ml) with 120 mg palladium/C was hydrogenated overnight at room temperature using a balloon to give 2.00 g (100%) of the title compound.

### Step F

### 1-[4-(tert-Butyldimethylsilanyloxy)phenyl]-2-(2,4-dichlorophenyl)-5-methyl-1H-imidazole-4-carboxylic acid (4,4-difluorocyclohexyl)amide

To a solution of 2-(2,4-dichlorophenyl)-1-(4-hydroxyphenyl)-5-methyl-1H-imidazole-4-carboxylic acid (4,4-difluorocyclohexyl)amide (2.00 g, 4.16 mmol) in dichloromethane (100 ml) was added imidazole (1.13 g, 16.6 mmol) followed by t-butyldimethylchlorosilane (2.50 g, 16.6 mmol). The reaction mixture was stirred at room temperature overnight, diluted with water and extracted with dichloromethane (×2). Drying (Na₂SO₄), filtration and concentration followed by chromatography (heptane:EtOAc gradient) afforded 2.20 g (88%) of the title compound as a colorless solid.

### Step G

### 5-Bromomethyl-1-[4-(tert-Butyldimethylsilanyloxy)phenyl]-2-(2,4-dichlorophenyl)-1H-imidazole-4-carboxylic acid (4,4-difluorocyclohexyl)-amide

To a solution of 1-[4-(tert-butyldimethylsilanyloxy)phenyl]-2-(2,4-dichlorophenyl)-5-methyl-1H-imidazole-4-carboxylic acid (4,4-difluorocyclohexyl)-amide (0.93 g, 1.56 mmol) in 1,2-dichloroethane (30 ml) was added N-bromosuccinimide (0.42 g, 2.36 mmol) and AIBN (cat.). The reaction mixture was heated at reflux for 2 hours, concentrated and purified by flash chromatography (heptane:EtOAc gradient) to give 0.80 g (76%) of the title compound as a colorless solid.

### Step H

### 2-(2,4-Dichlorophenyl)-5-hydroxymethyl-1-(4-hydroxyphenyl)-1H-imidazole-4-carboxylic acid (4,4-difluorocyclohexyl)amide

To a solution of 5-bromomethyl-1-[4-(tert-Butyldimethylsilanyloxy)phenyl]-2-(2,4-dichlorophenyl)-1H-imidazole-4-carboxylic acid (4,4-difluorocyclohexyl)amide (0.80 g, 1.19 mmol) in acetone:water (20 ml: 20 ml) was added silver nitrate (0.70 g, 4.12 mmol) and the reaction mixture stirred at 60° C. overnight, cooled to room temperature and filtered. Water was added and the product extracted with dichloromethane (×2). Drying (Na₂SO₄), filtration, concentration and chromatography gave 270 mg of the title compound together with 260 mg 1-[4-(tert-butyl-dimethylsilanyloxy)phenyl]-2-(2,4-dichlorophenyl)-5-hydroxymethyl-1H-imidazole-4-carboxylic acid (4,4-difluorocyclohexyl)amide. The latter was transformed to the title product with HF in acetonitrile. In total 470 mg (79%) of the title compound was obtained as a colorless solid.

### Step I

### 3,3,3-Trifluoropropane-1-sulfonic acid 4-[2-(2,4-dichlorophenyl)-4-(4,4-difluoro-cyclohexylcarbamoyl)-5-hydroxymethylimidazol-1-yl]phenyl ester

To a solution of 2-(2,4-dichlorophenyl)-5-hydroxymethyl-1-(4-hydroxyphenyl)-1H-imidazole-4-carboxylic acid (4,4-difluorocyclohexyl)-amide (470 mg, 0.95 mmol) in dichloromethane (20 ml) was added triethylamine (0.15 ml, 1.05 mmol) at 0° C., followed by 3,3,3-trifluoropropanesulfonyl chloride (204 mg, 1.05 mmol). The reaction mixture was stirred at 0° C. for 2 hours, poured onto water and the aqueous phase extracted with DCM (×2). Drying (Na₂SO₄), filtration and concentration followed by chromatography (heptane: EtOAc 70: 30-50:50) gave 400 mg (65%) of the title compound as a colorless solid.

HPLC: 79%. MS: 678 (M+Na)

### Example 4

### 3,3,3-Trifluoropropane-1-sulfonic acid 4-[4-cyclohexylcarbamoyl-2-(2,4-dichlorophenyl)-5-hydroxymethylimidazol-1-yl]-phenyl ester

### Step A 2-(2,4-Dichlorophenyl)-5-hydroxymethyl-1-(4-methoxyphenyl)-1H-imidazole-4-s carboxylic acid

To a solution of 5-bromomethyl-2-(2,4-dichlorophenyl)-1-(4-methoxyphenyl)-1H-imidazole-4-carboxylic acid ethyl ester, prepared as in Ex. 1, Step D (3.68 g, 7.60 mmol) in THF (30 ml) was added a 5% solution of sodium hydroxide in water (30 ml). The reaction mixture was boiled at reflux for 3 hours, cooled to 0° C., acidified with 1M HCl to pH 5. Extraction with EtOAc (×3), washing of the combined organic extracts with brine, drying (Na₂SO₄), filtration and concentration gave 2.80 g (93%) of the title compound as a colorless solid.

### Step B 2-(2,4-Dichlorophenyl)-5-hydroxymethyl-1-(4-methoxyphenyl)-1H-imidazole-4-carboxylic acid cyclohexylamide

To a solution of 2-(2,4-dichlorophenyl)-5-hydroxymethyl-1-(4-methoxyphenyl)-1H-imidazole-4-carboxylic acid (1.18 g, 3.00 mmol) in DMF (40 ml) was added triethylamine (1.05 ml, 7.50 mmol), cyclohexylamine (0.35 ml, 3.00 mmol) and BOP (1.60 g, 3.60 mmol). The reaction mixture was stirred at room temperature overnight, poured into ice-water and extracted with EtOAc (×2). The combined organic extracts were washed with brine, dried (Na₂SO₄), filtered and concentrated. Flash chromatography (heptane:EtOAc gradient) afforded 530 mg (37%) of the product as a colorless solid.

Step C₂-(2,4-Dichlorophenyl)-5-hydroxymethyl-1-(4-hydroxyphenyl)-1H-imidazole-4-carboxylic acid cyclohexylamide To as solution of 2-(2,4-dichlorophenyl)-5-hydroxymethyl-1-(4-methoxyphenyl)-1H-imidazole-4-carboxylic acid cyclohexylamide (530 mg, 1.11 mmol) in dichloromethane (20 ml) was added BBr₃ (0.43 ml, 4.44 mmol) at 0° C. The reaction mixture was stirred for 2.5 hrs at room temperature, 1 M HCl added and the product extracted with EtOAc (×2). The organic extract was dried (Na₂SO₄), filtered, concentrated and purified by flash chromatography (heptane:EtOAc 50:50-EtOAc) to give 300 mg (59%) of the product as a colorless solid.

### Step D 3,3,3-Trifluoropropane-1-sulfonic acid 4-[4-cyclohexylcarbamoyl-2-(2,4-dichloro-phenyl)-5-hydroxymethyl-imidazol-1-yl]-phenyl ester

To a magnetically stirred solution of 2-(2,4-dichlorophenyl)-5-hydroxymethyl-1-(4-hydroxy-phenyl)-1H-imidazole-4-carboxylic acid cyclohexylamide (300 mg, 0.65 mmol) in dry dichloromethane (20 ml) at 0° C. was added triethylamine (90 μl, 0.65 mmol) followed by 3,3,3-trifluoro-1-propane sulfonyl chloride (128 mg, 0.65 mmol). The cooling bath was removed and the reaction mixture stirred for 1.5 hours at room temperature. The reaction mixture was concentrated and purified by flash chromatography (heptane:EtOAc gradient) to afford 150 mg (38%) of the title compound as a colorless solid.

HPLC: 95%

MS (M+Na): 643

### Example 5

### 3,3,3-Trifluoropropane-1-sulfonic acid 4-[2-(2,4-dichlorophenyl)-4-(cis-2-hydroxy-cyclohexylcarbamoyl)-5-hydroxymethyl-imidazol-1-yl]-phenyl ester

### Step A 2-(2,4-Dichlorophenyl)-5-hydroxymethyl-1-(4-methoxyphenyl)-1H-imidazole-4-carboxylic acid (2-hydroxycyclohexyl)-amide

To a solution of 2-(2,4-dichlorophenyl)-5-hydroxymethyl-1-(4-methoxyphenyl)-1H-imidazole-4-carboxylic acid, prepared as in Ex. 1, Step D (1.30 g, 3.30 mmol) in DMF (50 ml) was added triethylamine (0.90 ml, 6.50 mmol), cis-2-aminocyclohexanol hydrochloride (0.50 g, 3.31 mmol) and BOP (1.80 g, 4.07 mmol). The reaction mixture was stirred at room temperature overnight, poured into water and extracted with EtOAc (×3). The combined organic extracts were washed with brine, dried (Na₂SO₄), filtered and concentrated. Flash chromatography (heptane:EtOAc 50:50-EtOAc) afforded 0.63 g (39%) of the product as a colorless solid.

### Step B 2-(2,4-Dichlorophenyl)-5-hydroxymethyl-1-(4-hydroxyphenyl)-1H-imidazole-4-carboxylic acid (cis-2-hydroxycyclohexyl)-amide

To as solution of 2-(2,4-dichlorophenyl)-5-hydroxymethyl-1-(4-methoxyphenyl)-1H-imidazole-4-carboxylic acid (2-hydroxycyclohexyl)amide (0.63 g, 1.28 mmol) in dichloromethane (20 ml) was added BBr₃ (0.50 ml, 5.20 mmol) at 0° C. The reaction mixture was stirred for 2 hrs at room temperature, 1 M HCl added and the product extracted with EtOAc (×2). The organic extract was dried (Na₂SO₄), filtered, concentrated and purified by flash chromatography (heptane:EtOAc 50:50-EtOAc) to give 410 mg (68%) of the product as a colorless solid.

### Step C 3,3,3-Trifluoropropane-1-sulfonic acid 4-[2-(2,4-dichlorophenyl)-4-(cis-2-hydroxy-cyclohexylcarbamoyl)-5-hydroxymethyl-imidazol-1-yl]-phenyl ester

To a magnetically stirred solution of 2-(2,4-dichlorophenyl)-5-hydroxymethyl-1-(4-hydroxy-phenyl)-1H-imidazole-4-carboxylic acid (2-hydroxycyclohexyl)amide (400 mg, 0.84 mmol) in dry dichloromethane (20 ml) at 0° C. was added triethylamine (0.23 ml, 1.68 mmol) followed by 3,3,3-trifluoro-1-propane sulfonyl chloride (165 mg, 0.84 mmol). The cooling bath was removed and the reaction mixture stirred for 2 hours at room temperature. The reaction mixture was concentrated and purified by flash chromatography (heptane: EtOAc 70:30-50:50:40:60) to afford 230 mg (43%) of the title compound as a colorless solid.

HPLC: 94%

MS (M+Na): 658

The following compounds are prepared as described as above:

### Ex. 6

3,3,3-Trifluoropropane-1-sulfonic acid 4-[2-(3-cyano-5-fluoro-phenyl)-4-(4,4-difluoro-cyclohexylcarbamoyl)-5-hydroxymethylimidazol-1-yl]phenyl ester

### Ex. 7

3,3,3-Trifluoropropane-1-sulfonic acid 4-[2-(3-cyanophenyl)-5-hydroxymethyl-4-(1-hydroxymethyl-3-methylbutylcarbamoyl)imidazol-1-yl]phenyl ester

### Ex. 8

3,3,3-Trifluoro-propane-1-sulfonic acid 4-[4-(2-aminocyclohexylcarbamoyl)-2-(3-cyano-5-fluoro-phenyl)-5-hydroxymethylimidazol-1-yl]phenyl ester

### Ex. 9

3,3,3-Trifluoropropane-1-sulfonic acid 4-[2-(3-cyano-5-fluoro-phenyl)-4-(3-dimethylamino-cyclohexylcarbamoyl)-5-hydroxymethylimidazol-1-yl]phenyl ester

### Ex. 10

3,3,3-Trifluoropropane-1-sulfonic acid 4-[2-(3-cyano-5-fluoro-phenyl)-4-(2-hydroxy-cyclohexylcarbamoyl)-5-hydroxymethylimidazol-1-yl]phenyl ester

### Ex. 11

3,3,3-Trifluoropropane-1-sulfonic acid 4-[2-(3-cyanophenyl)-4-(2-hydroxy-cyclohexylcarbamoyl)-5-hydroxymethylimidazol-1-yl]phenyl ester

### Ex. 12

3,3,3-Trifluoropropane-1-sulfinic acid 4-[2-(3-cyano-5-fluorophenyl)-4-(3-hydroxy-cyclohexylcarbamoyl)-5-hydroxymethyl-imidazol-1-yl]phenyl ester

### Ex. 13

2-(2,4-Dichlorophenyl)-1-[4-(3-fluoropropoxy)phenyl]-5-hydroxymethyl-1H-imidazole-4-carboxylic acid cyclohexylamide

### Ex. 14

3,3,3-Trifluoropropane-1-sulfonic acid 4-[2-(2-chlorophenyl)-4-(2-hydroxy-cyclohexylcarbamoyl)-5-hydroxymethylimidazol-1-yl]phenyl ester

### Ex. 15

3,3,3-Trifluoropropane-1-sulfonic acid 4-[2-(2-chlorophenyl)-4-(1,4-dimethyl-pentylcarbamoyl)-5-hydroxymethyl-imidazol-1-yl]phenyl ester

### Ex. 16

2-(2,4-Dichlorophenyl)-5-hydroxymethyl-1-[4-(3,3,3-trifluoropropoxy)phenyl]-1H-imidazole-4-carboxylic acid (2-hydroxy-cyclohexyl)amide

### Ex. 17

3,3,3-Trifluoropropane-1-sulfonic acid 4-[2-(4-chloro-2-methyl-phenyl)-5-hydroxymethyl-4-(piperidin-1-ylcarbamoyl)-imidazol-1-yl]phenyl ester

### Ex. 18

2-(2,4-Dichloro-phenyl)-5-hydroxymethyl-1-[4-(3,3,3-trifluoropropoxy)phenyl]-1H-imidazole-4-carboxylic acid (3-hydroxycyclohexyl)amide

### Ex. 19

3-Fluoro-propane-1-sulfonic acid 4-[2-(2,4-dichlorophenyl)-4-(2-hydroxy-cyclohexylcarbamoyl)-5-hydroxymethylimidazol-1-yl]-phenyl ester

### Ex. 20

4,4,4-Trifluorobutane-1-sulfonic acid 4-[2-(3-cyano-5-fluoro-phenyl)-4-(1-ethyl-butylcarbamoyl)-5-hydroxymethyl-imidazol-1-yl]phenyl ester

### Ex. 21

3,3,3-Trifluoropropane-1-sulfonic acid 4-[2-(2-chlorophenyl)-4-cyclohexyl-carbamoyl-5-hydroxymethylimidazol-1-yl]phenyl ester

1. A compound of formula (I) 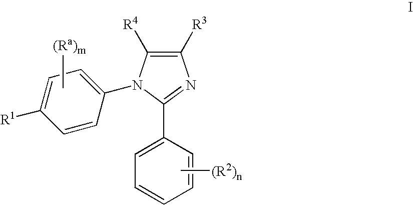
or a pharmaceutically acceptable salt thereof, in which
R¹ represents a) a C₁₋₆alkoxy group optionally substituted by one or more fluoro b) a group of formula phenyl(CH₂)ₚO— in which p is 1, 2 or 3 and the phenyl ring is optionally substituted by 1, 2 or 3 groups represented by Z, c) a group R⁵S(O)₂O or R⁵S(O)₂NH in which R⁵ represents a C₁₋₆alkyl group optionally substituted by one or more fluoro, or R⁵ represents phenyl or a heteroaryl group each of which is optionally substituted by 1, 2 or 3 groups represented by Z or d) a group of formula (R⁶)₃ Si in which R⁶ represents a C₁₋₆alkyl group which may be the same or different;
R^{a} represents halo, a C₁₋₃alkyl group or a C₁₋₃alkoxy group;
m is 0, 1, 2 or 3;
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸
in which X is CO or SO₂,
Y is absent or represents NH optionally substituted by a C₁₋₃alkyl group;
and R⁷ and R⁸ independently represent:
a C₁₋₉alkyl group optionally substituted by 1, 2, or 3 groups represented by W;
a C₃₋₁₅cycloalkyl group optionally substituted by 1, 2, or 3 groups represented by W;
an optionally substituted (C₃₋₅cycloalkyl)C₁₋₃alkylene group optionally substituted by 1, 2, or 3 groups represented by W;
a group —(CH₂)ₛ(phenyl) in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜ Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl; or
b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents carboxy, a C₁₋₃alkoxycarbonyl group, hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl.

2. The compound as claimed in claim 1 in which
R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O in which R⁵ represents a C₁₋₆alkyl group optionally substituted by one or more fluoro, or R⁵ represents phenyl or a heteroaryl group each of which is optionally substituted by 1, 2 or 3 groups represented by Z;
R^{a} represents halo, a C₁₋₃alkyl group or a C₁₋₃alkoxy group;
m is 0, 1, 2 or 3;
R² represents halo
n is 0, 1, 2 or 3;
R³ represents
a) a group X—Y—NR⁷R⁸
in which X is CO;
Y is absent or represents NH optionally substituted by a C₁₋₃alkyl group;
and R⁷ and R⁸ independently represent:
a C₁₋₆alkyl group optionally substituted by 1,2, or 3 groups represented by W;
a C₃₋₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂) phenyl), in which r is 0, 1, 2, 3 or 4, s is I when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜ Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group,
a C₁₋₅alkoxy group or halo;
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl;
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino, C₁₋₃alkylsulphonyl, C₁₋₃alkoxycarbonyl, carboxy, cyano, carbamoyl, mono or di C₁₋₃alkyl carbamoyl and acetyl; and
W represents hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl.

3. The compound of formula I according to claim 1 as represented by formula IA 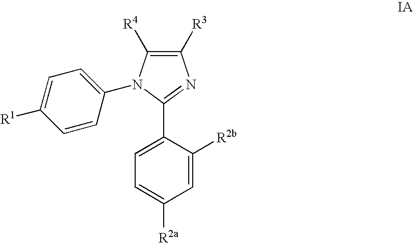
in which
R¹ is a) a C₃₋₆alkoxy group substituted by one or more fluoro, b) a group of formula phenyl(CH₂)ₚO— in which p is 1, 2 or 3 and the phenyl ring is optionally substituted by 1, 2 or 3 groups represented by Z, c) a group R⁵S(O)₂O in which R⁵ represents a C₁₋₆alkyl group optionally substituted by one or more fluoro;
R^{2a} represents H or chloro;
R^{2b} represents H or chloro;
R³ represents a group CONHNR⁷R⁸ in which NR⁷R⁸ represents piperidino; and
R⁴ represents a C₁₋₆alkyl group substituted by one or more of the following: hydroxy, a group NR^{e}R^{f} in which R^{e} and R^{f} independently represent H, a C₁₋₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group.

4. The compound of formula I according to claim 1 as represented by formula IA 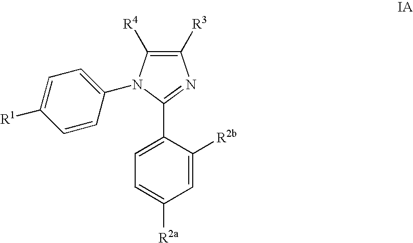
in which
R¹ is a) a C₃₋₆alkoxy group substituted by one or more fluoro or b) a group R⁵S(O)₂O in which R⁵ represents a C₁₋₆alkyl group optionally substituted by one or more fluoro, or R⁵ represents phenyl or a heteroaryl group each of which is optionally substituted by 1, 2 or 3 groups represented by Z;
R^{2a} represents chloro;
R^{2b} represents chloro;
R³ represents a group CONHNR⁷R⁸ in which NR⁷R⁸ represents piperidino; and
R⁴ represents a hydroxymethyl group.

5. The compound of formula (I) according to claim 1 as represented by formula IB 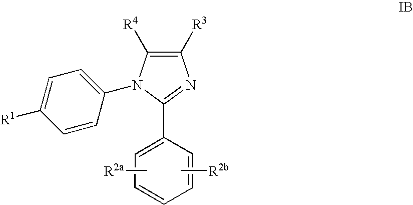
in which
R¹ is a) a C₃₋₆alkoxy group substituted by one or more fluoro or b) a group R⁵S(O)₂O in which R⁵ represents a C₁₋₆alkyl group optionally substituted by one or more fluoro;
R^{2a} represents H, a C₁₋₃alkyl group, chloro, fluoro or cyano;
R^{2b} represents a C₁₋₃alkyl group, chloro, fluoro or cyano;
R³ represents a group CONHNR⁷R⁸ in which NR⁷R⁸ represents piperidino or R³ represents a group CONHR⁸ in which R⁸ represents a cycloalkyl group optionally substituted by one or more of the following: fluoro, hydroxy, a group NR^{e}R^{f} in which R^{e} and R^{f} independently represent H or a C₁₋₆alkyl group or R⁸ represents a C₅₋₈alkyl group optionally substituted by hydroxy; and
R⁴ represents a C₁₋₃alkyl group substituted at the terminal carbon by hydroxy.

6. The compound as claimed in claim 1 in which R¹ represents a group R⁵S(O)₂O in which R⁵ represents a C₁₋₆alkyl group optionally substituted by one or more fluoro.

7. The compound as claimed in claim 1 in which R³ represents a group CONHNR⁷R⁸ in which NR⁷R⁸ represents piperidino.

8. The compound as claimed in claim 1 in which R¹ is a C₃₋₆alkoxy group substituted by one or more fluoro.

9. The compound as claimed in claim in which R¹ is a group of formula phenyl(CH₂)ₚO— in which p is 1, 2 or 3.

10. The compound as claimed in claim 1 in which R¹ is a group R⁵S(O)₂O in which R⁵ represents a C₁₋₆alkyl group substituted by one or more fluoro.

11. The compound according to claim 9 in which R¹ represents a group R⁵S(O)₂O in which R⁵ represents a C₃₋₆alkyl group substituted by one or more fluoro.

12. The compound as claimed in claim 1 selected from:
propane-1-sulfonic acid 4-[2-(2,4-dichlorophenyl)-5-hydroxymethyl-4-(piperidin-1-ylcarbamoyl)-imidazol-1-yl]phenyl ester;
3,3,3-trifluoropropane-1-sulfonic acid 4-[2-(2,4-dichlorophenyl)-5-hydroxymethyl-4-(piperidin-1-ylcarbamoyl)imidazol-1-yl]phenyl ester;
3,3,3-trifluoropropane-1-sulfonic acid 4-[2-(2,4-dichlorophenyl)-4-(4,4-difluoro-cyclohexylcarbamoyl)-5-hydroxymethylimidazol-1-yl]phenyl ester;
3,3,3-trifluoropropane-1-sulfonic acid 4-[4-cyclohexylcarbamoyl-2-(2,4-dichlorophenyl)-5-hydroxymethylimidazol-1-yl]-phenyl ester;
3,3,3-trifluoropropane-1-sulfonic acid 4-[2-(2,4-dichlorophenyl)-4-(cis-2-hydroxy-cyclohexylcarbamoyl)-5-hydroxymethyl-imidazol-1-yl]-phenyl ester;
3,3,3-trifluoropropane-1-sulfonic acid 4-[2-(3-cyano-5-fluoro-phenyl)-4-(4,4-difluoro-cyclohexylcarbamoyl)-5-hydroxymethylimidazol-1-yl]phenyl ester;
3,3,3-trifluoropropane-1-sulfonic acid 4-[2-(3-cyanophenyl)-5-hydroxymethyl-4-(1-hydroxymethyl-3-methylbutylcarbamoyl)imidazol-1-yl]phenyl ester;
3,3,3-trifluoro-propane-1-sulfonic acid 4-[4-(2-aminocyclohexylcarbamoyl)-2-(3-cyano-5-fluoro-phenyl)-5-hydroxymethylimidazol-1-yl]phenyl ester;
3,3,3-trifluoropropane-1-sulfonic acid 4-[2-(3-cyano-5-fluoro-phenyl)-4-(3-dimethylamino-cyclohexylcarbamoyl)-5-hydroxymethylimidazol-1-yl]phenyl ester;
3,3,3-trifluoropropane-1-sulfonic acid 4-[2-(3-cyano-5-fluoro-phenyl)-4-(2-hydroxy-cyclohexylcarbamoyl)-5-hydroxymethylimidazol-1-yl]phenyl ester;
3,3,3-trifluoropropane-1-sulfonic acid 4-[2-(3-cyanophenyl)-4-(2-hydroxy-cyclohexylcarbamoyl)-5-hydroxymethylimidazol-1-yl]phenyl ester;
3,3,3-trifluoropropane-1-sulfinic acid 4-[2-(3-cyano-5-fluorophenyl)-4-(3-hydroxy-cyclohexylcarbamoyl)-5-hydroxymethyl-imidazol-1-yl]phenyl ester;
2-(2,4-dichlorophenyl)-1-[4-(3-fluoropropoxy)phenyl]-5-hydroxymethyl-1H-imidazole-4-carboxylic acid cyclohexylamide;
3,3,3-trifluoropropane-1-sulfonic acid 4-[2-(2-chlorophenyl)-4-(2-hydroxy-cyclohexylcarbamoyl)-5-hydroxymethylimidazol-1-yl]phenyl ester;
3,3,3-trifluoropropane-1-sulfonic acid 4-[2-(2-chlorophenyl)-4-(1,4-dimethyl-pentylcarbamoyl)-5-hydroxymethyl-imidazol-1-yl]phenyl ester;
2-(2,4-dichlorophenyl)-5-hydroxymethyl-1-[4-(3,3,3-trifluoropropoxy)phenyl]-1H-imidazole-4-carboxylic acid (2-hydroxy-cyclohexyl)amide;
3,3,3-trifluoropropane-1-sulfonic acid 4-[2-(4-chloro-2-methyl-phenyl)-5-hydroxymethyl-4-(piperidin-1-ylcarbamoyl)-imidazol-1-yl]phenyl ester;
2-(2,4-dichlorophenyl)-5-hydroxymethyl-1-[4-(3,3,3-trifluoropropoxy)phenyl]-1H-imidazole-4-carboxylic acid (3-hydroxycyclohexyl)amide;
3-fluoro-propane-1-sulfonic acid 4-[2-(2,4-dichlorophenyl)-4-(2-hydroxy-cyclohexylcarbamoyl)-5-hydroxymethylimidazol-1-yl]phenyl ester;
4,4,4-trifluorobutane-1-sulfonic acid 4-[2-(3-cyano-5-fluorophenyl)-4-(1-ethyl-butylcarbamoyl)-5-hydroxymethyl-imidazol-1-yl]phenyl ester; and
3,3,3-trifluoropropane-1-sulfonic acid 4-[2-(2-chlorophenyl)-4-cyclohexylcarbamoyl-5-hydroxymethylimidazol-1-yl]phenyl ester;
and pharmaceutically acceptable salts thereof.

13. (canceled)

14. A pharmaceutical formulation comprising a compound of formula I according to claim 1 or claim 12 and a pharmaceutically acceptable adjuvant, diluent or carrier.

15. (canceled)

16. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound of formula I as claimed in claim 1 to a patient in need thereof.

17. (canceled)

18. A process for the preparation of a compound of formula (I) as claimed in claim 1 in which R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O in which R⁵ is as defined in claim 1 comprising reacting a compound of formula II 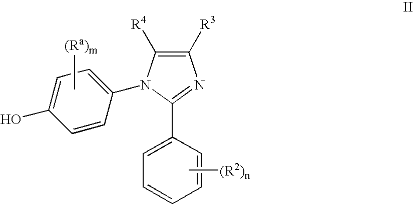
in which R², R³, R⁴, R^{a}, m and n are as defined in claim 1 with a group R^{1A}-X in which R^{1A} represents a group such that R^{1A}O represents R¹ in which R¹ represents a) a C₃₋₆alkoxy group substituted by one or more fluoro or b) a group of formula phenyl(CH₂)ₚO— in which p is 1, 2 or 3 and the phenyl ring is optionally substituted by 1, 2 or 3 groups represented by Z, or c) a group R⁵S(O)₂O in which R⁵ is as defined in claim 1 and X represents a leaving group at a temperature in the range of −25 to 150° C., in the presence of an inert solvent and optionally in the presence of a base.

19. A compound of formula II 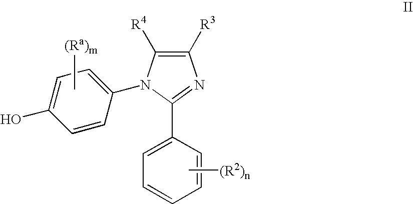
in which
R^{a} represents halo, a C₁₋₃alkyl group or a C₁₋₃alkoxy group;
m is 0, 1, 2 or 3:
R² represents a C₁₋₃alkyl group, a C₁₋₃alkoxy group, hydroxy, nitro, cyano or halo
n is 0, 1, 2 or 3;
R³ represents
a) a group X—Y—NR⁷R⁸
in which X is CO or SO₂,
Y is absent or represents NH optionally substituted by a C₁₋₃alkyl group;
and R⁷ and R⁸ independently represent:
a C₁₋₉alkyl group optionally substituted by 1, 2, or 3 groups represented by W;
a C₃₋₁₅cycloalkyl group optionally substituted by 1, 2, or 3 groups represented by W;
an optionally substituted (C₃₋₁₅cycloalkyl)C₁₋₃alkylene group optionally substituted by 1, 2, or 3 groups represented by W;
a group —(CH₂)ᵣ(phenyl)ₛ in which r is 0, 1, 2, 3 or 4, s is 1 when r is 0 otherwise s is 1 or 2 and the phenyl groups are optionally independently substituted by one, two or three groups represented by Z;
a saturated 5 to 8 membered heterocyclic group containing one nitrogen and optionally one of the following: oxygen, sulphur or an additional nitrogen wherein the heterocyclic group is optionally substituted by one or more C₁₋₃alkyl groups, hydroxy or benzyl;
a group —(CH₂)ₜ Het in which t is 0, 1, 2, 3 or 4, and the alkylene chain is optionally substituted by one or more C₁₋₃alkyl groups and Het represents a heteroaryl group optionally substituted by one, two or three groups selected from a C₁₋₅alkyl group, a C₁₋₅alkoxy group or halo:
or R⁷ represents H and R⁸ is as defined above;
or R⁷ and R⁸ together with the nitrogen atom to which they are attached represent a saturated or partially unsaturated 5 to 8 membered heterocyclic group containing one nitrogen and optionally one of the following: oxygen, sulphur or an additional nitrogen; wherein the heterocyclic group is optionally substituted by one or more C₁₋₃alkyl groups, hydroxy, fluoro or benzyl; or
b) oxazolyl, isoxazolyl, thiazolyl, isothiazolyl, oxadiazolyl, thiadiazolyl, pyrrolyl, pyrazolyl, imidazolyl, triazolyl, tetrazolyl, thienyl, furyl or oxazolinyl, each optionally substituted by 1, 2 or 3 groups Z;
R⁴ represents a C₁₋₆alkyl group substituted by one or more of the following: hydroxy, a group NR^{e}R^{f} in which R^{e} and R^{f} independently represent H, a C₆alkyl group optionally substituted by one or more hydroxy or one or more C₁₋₆alkoxy groups or R^{e} and R^{f} together with the nitrogen to which they are attached represent a 4 to 7 membered saturated heterocyclic ring optionally containing an oxygen or a second nitrogen wherein said ring is optionally substituted by one or more of the following: hydroxy, fluoro or a C₁₋₆alkyl group;
Z represents a C₁₋₃alkyl group, a C₁₋₃alkoxy group, hydroxy, halo, trifluoromethyl, trifluoromethylthio, difluoromethoxy, trifluoromethoxy, trifluoromethylsulphonyl, nitro, amino, mono or di C₁₋₃alkylamino alkylsulphonyl, C₁₋₃alkoxycarbonyl, carboxy, cyano, carbamoyl, mono or di C₁₋₃alkyl carbamoyl and acetyl; and
W represents carboxy, a C₁₋₃alkoxycarbonyl group, hydroxy, fluoro, a C₁₋₃alkyl group, a C₁₋₃alkoxy group, amino, mono or di C₁₋₃alkylamino, or a heterocyclic amine selected from morpholinyl, pyrrolidinyl, piperidinyl or piperazinyl in which the heterocyclic amine is optionally substituted by a C₁₋₃alkyl group or hydroxyl.
